(19) United States
(12) Patent Application Publication
(10) Pub. No.: US 2026/0216116 A1
(43) Pub. Date: Jul. 30, 2026

(54) QUATERNARY AMMONIUM COMPOUNDS (QACS) AND PROCESS FOR PREPARATION THEREOF

(71) Applicants: Rajaram SAMANT, Thane, Maharashtra (IN); Manoj TONGRA, Thane, Maharashtra (IN)

(72) Inventors: Rajaram SAMANT, Thane (IN); Manoj TONGRA, Thane (IN); Jotiram PALKAR, Thane (IN)

(21) Appl. No.: 19/143,335

(22) PCT Filed: Nov. 6, 2023
(86) PCT No.: PCT/IN2023/051022

§ 371 Date: Jun. 25, 2025

(30) Foreign Application Priority Data
Jan. 3, 2023 (IN) 202321000328

(51) Int. Cl. A61K 31/205; C07C 53/10; C07C 63/08; C07C 229/12; C07C 229/18

(52) U.S. Cl. CPC A61K 31/205; C07C 53/10; C07C 63/08; C07C 229/12; C07C 229/18

(57) ABSTRACT

The present invention disclosed herein relates to a quaternary ammonium compound represented by Formula-I and simple, viable process for preparation thereof. Particularly, the present invention relates to novel therapeutically active quaternary ammonium carboxylate compounds. The compounds of Formula-I are useful for treating metabolic, neuropsychiatric, neurodevelopmental conditions, pregnancy health, fetal growth, cognitive development.

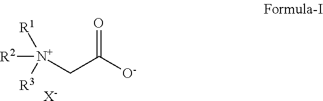

### FIELD OF THE INVENTION

The present invention relates to a quaternary ammonium compound of Formula-I, and a simple and viable process for preparation thereof. Particularly, the present invention relates to novel therapeutically active quaternary ammonium carboxylate compounds. The present novel molecular compounds of Formula-I are useful for treating metabolic, neuropsychiatric, neurodevelopmental conditions, pregnancy health, fetal growth and cognitive development.

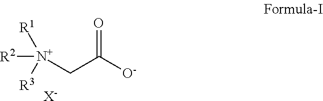

### BACKGROUND OF THE INVENTION

Quaternary ammonium compounds, also called as Quaternary Ammonium Cations or QACs, were first introduced as derivatives of hexamethylene tetramine, and the bactericidal properties of these salts were reported in several publications since 1915. QACs are some of the most extensively used classes of biocides, disinfectants, sanitizers, antimicrobials, and cleaners. QACs are widely applied in household, food-processing, agriculture, and clinical settings to control the spread of environmentally transmitted pathogens.

In chemistry, quaternary ammonium cations, also known as Quats, are positively charged polyatomic ions of the structure NR⁺⁴, R being an alkyl group or an aryl group. Unlike the ammonium ion (NH⁺⁴) and the primary, secondary, or tertiary ammonium cations, the quaternary ammonium cations are permanently charged, independent of the pH of their solution.

Quats are strong compounds, characterized by a fast, long-lasting effect against common gram+ and gram− bacteria, fungi, algae and enveloped viruses such as HIV, influenza, herpes and corona.

Quats are miscible with water or lower alcohols such as methanol, ethanol and propanol. The length of the alkyl chain length affects the different properties of Quats. A shorter alkyl chain length is associated with higher antimicrobial activity but higher levels of skin sensitivity, as well as greater solubility in water.

EP1125927B1 discloses quaternary ammonium salts as starting materials for medicines and quasi-drugs and cosmetics, phase-transfer catalysts, ionized solvents, etc.

Levocarnitine is a quaternary ammonium compound used to treat carnitine deficiency or to stimulate gastric and pancreatic secretions in hyperlipoproteinemia.

DE19839443A1 describes pharmaceutical and/or dietetic product contains a quaternary ammonium compound and/or S-adenosyl-methionine (SAM) for the prevention and treatment of disorders and damage caused by a deficient oxygen supply or of side effects of anti-inflammatory drugs as well as for the prevention and treatment of energy metabolism disorders. EP2521711B1 discloses prodrug delivery systems for tertiary amine-containing drugs, wherein the parent drug is asenapine or olanzapine.

WO2013179244A2 relates to use of quaternary halogenated ammonium salts for treating leishmaniasis infections and other parasitic diseases is described. It is further reported the use of Quats containing a halogenated N-methyl substituent as a therapeutic agent against leishmaniasis.

U.S. Pat. No. 8,846,937 B2 provides mono-Quaternary Ammonium Compounds which are modulators of nicotinic acetylcholine receptors for the prevention and/or treatment of central nervous system disorders, substance use and/or abuse, and or gastrointestinal tract disorders.

Further U.S. Pat. No. 7,687,546 B2 relates to a novel quaternary ammonium compound, a process for producing the same, and a therapeutic agent for cerebrovascular disorder and a therapeutic agent for heart disease having said quaternary ammonium compound as an active ingredient thereof.

Endogenous quaternary ammonium compounds including acetylcholine, choline, carnitine, acetylcarnitine and seven other acylcarnitines of low polarity are involved in various physiological processes in the central nervous system.

It is known that quaternary amines are an extremely important in various classes of compounds from drug discovery. Many of these drugs are useful in therapeutic areas such as for their effects on the central nervous system (CNS) of a patient that would benefit from sustained release formulations.

Carboxylic acids are often present as a functional group of effective medicines. They are involved in specific charge-charge interactions and are thus often critical for the binding of agents to their targets. The carboxylic acid functional group adds to the hydrophilicity of the drug as well as to its polarity and this may impede the bioavailability. Most carboxylic acids have a pKa value of about 3.5 to 4.5 and thus these compounds are ionized (deprotonated) under physiological conditions.

These properties of carboxylic acids in active principles have been well recognized for many years and a number of practical solutions have been identified with the most common one being that of ester modification.

Therefore, the present inventors concentrate on this successful prodrug strategies for carboxylates, that cover prodrugs of Quats. More specifically, the inventors have developed novel molecular entity where Quats are stabilized with carboxylates that make the quats generally safe for administration with improved bioavailability.

### OBJECTIVES OF THE INVENTION

The primary object of the present invention is to provide a novel therapeutically active Quaternary Ammonium Carboxylate Compounds (QACs).

Another object of the present invention is to provide cost-effective, side-effect-free Quaternary Ammonium Carboxylate Compounds (QACs).

Yet another object of the present invention is to provide an improved and commercially viable process for preparation of novel Quaternary Ammonium Carboxylate Compounds (QACs) represented by Formula-I.

Another object of the present invention is to provide therapeutically active Quaternary Ammonium Carboxylate Compounds (QACs) with minimum side effects for treating metabolic disorders, neuropsychiatric disorders, neurodevelopmental conditions, pregnancy health complications, fetal growth complications and cognitive impairments.

### SUMMARY OF THE INVENTION

To meet the above objects, the inventors of the instant invention carried out thorough experiments to establish improved therapeutic efficacy of Quaternary Ammonium Carboxylate Compounds (QACs) in the treatment of metabolic disorders, neuropsychiatric disorders, pregnancy health complications, fetal growth complications and cognitive impairments.

In a particular aspect, the present invention relates to novel therapeutically active Quaternary Ammonium Carboxylate Compounds (QACs) represented by Formula-I and simple process for preparation thereof.

In another aspect, the present invention provides novel Quaternary Ammonium Carboxylate Compounds (QACs) represented by Formula-I with high purity and yield.

In another aspect, the present invention provides a commercially viable and non-hazardous process for the preparation of compounds represented by Formula-I.

In another aspect, the present invention provides therapeutically effective, non-toxic and safe Quaternary Ammonium Carboxylate Compounds (QACs) with no side effects.

### BRIEF DESCRIPTION OF FIGURES/DRAWINGS

FIGS. 1(a), 1(b) and 1(c) illustrates H₁ NMR DMSO (400 MHz, CD₃OD) of 1 carboxy N—N—N trimethylmethanaminium benzoate.

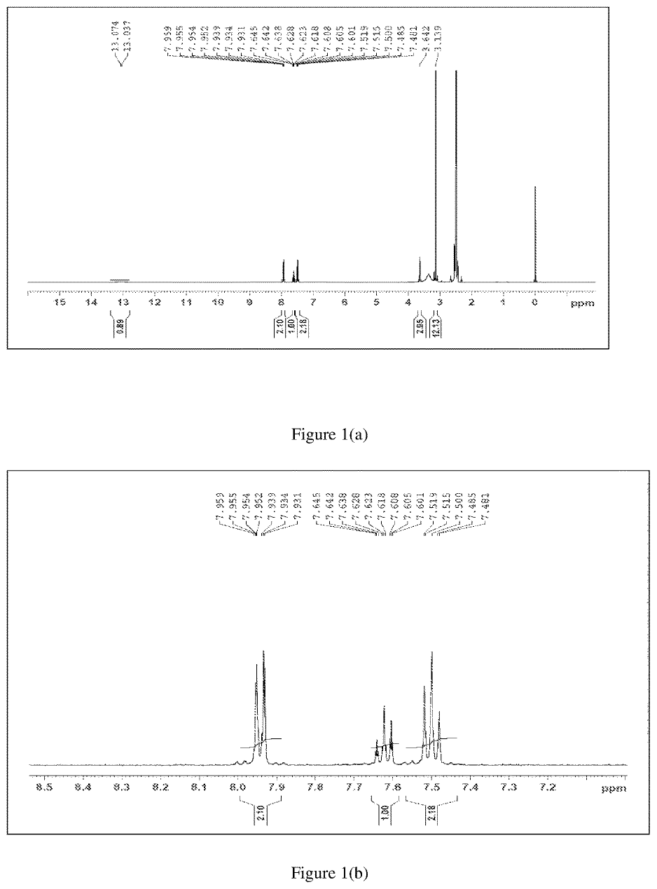

FIG. 2 illustrates HPLC chromatogram of benzoic acid.

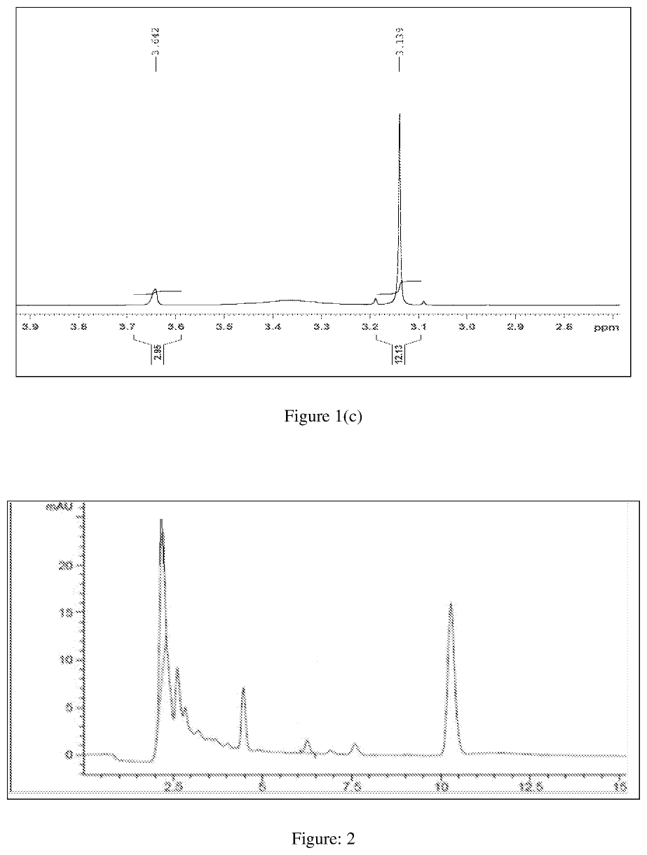

### DETAILED DESCRIPTION OF THE INVENTION

The invention will now be described in detail in connection with certain preferred and optional embodiments, so that various aspects thereof may be more fully interpreted and comprehended. However, any skilled person or artisan will appreciate the extent to which such embodiments could be generalized in practice.

It is further to be understood that all terminology used herein is for the purpose of describing particular embodiments only and is not intended to be limiting in any manner or scope.

Unless defined otherwise, all technical and scientific expressions used herein have the same meaning as commonly understood by one of ordinary skill in the art to which embodiments of the invention pertain.

In describing and claiming the embodiments of the present invention, the following terminology will be used in accordance with the definitions set out below which are known in the state of art.

The singular forms “a,” “an,” and “the” include plural reference unless the context clearly dictates otherwise. Also, the term ‘composition’ does not limit the scope of the invention for multiple compositions can be illustrated for best mode of the invention.

The term “pharmaceutically/nutraceutically acceptable salt,” as use herein, represents those salts which are within the scope of sound medical judgment, are suitable for use in contact with the tissues of humans and animals without undue toxicity, irritation, allergic response and the like and are commensurate with a reasonable benefit/risk ratio. Particularly, the term “pharmaceutically acceptable salts” refers to the relatively non-toxic, inorganic, and organic acid addition salts of compounds, alkali or alkaline earth metal salts, as well as solvates, co-crystals, polymorphs and the like of the salts.

All modifications and substitutions that come within the meaning of the description and the range of their legal equivalents are to be embraced within their scope. A description using the transitional phrase “comprising” allows the inclusion of other elements to be within the scope of the invention.

The term “Quaternary Ammonium Carboxylate Compounds (QACs)” is related to chemical compounds containing nitrogen atom which is attached to four hydrocarbon groups. Generally, QACs are any of a class of compounds in which the four hydrogen atoms of the ammonium radical are replaced by other groups preferably by organic radicals.

In one preferred embodiment, the invention relates to novel therapeutically active Quaternary Ammonium Carboxylate Compounds (QACs) represented by Formula-I:

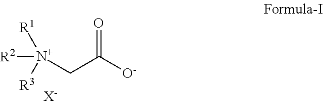

wherein R₁ R₂ and R₃ are similar or independently selected from the group consisting of H, OH, (C1-C9)alkyl, alkylene, alkyne, (C1-C9)-O—, heterocyclic group, —COOR (wherein R is lower alkyl, C1-C5), halide, substituted or unsubstituted cycloalkyl, aryl and arylalkyl;
wherein X is:

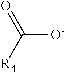

wherein R⁴ is selected from the group consisting of H, OH, (C1-C9)alkyl, alkylene, alkyne, aryl, aralkyl, (C3-C8) cycloalkyl, (C1-C9)-O—, heterocyclic group, —COOR (wherein R is lower alkyl, C1-C5), halide and

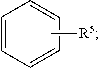

wherein R⁵ is selected from a group consisting of H, OH, alkyl, alkoxy, aryl, biphenyl, heterocyclic, cycloalkyl compounds.

In another embodiment, the present invention provides a novel Quaternary Ammonium Carboxylate Compounds (QACs) represented by Formula-I with a high bioavailability and therapeutic efficacy.

In another embodiment, the present invention provides a commercially feasible and simple process for the preparation of compounds represented by Formula-I comprising steps of: dehalogenation of QACs with compounds of Formula X in an organic solvent.

In another embodiment, the present invention provides simple process for preparation of compounds of Formula-I, comprising steps of:
i) dehalogenation quaternary ammonium compounds in presence of an organic solvent at warm condition to obtain crude quaternary ammonium carboxylate;
ii) purification of crude quaternary ammonium carboxylate compounds at suitable temperature to obtain high yield and purity compounds of Formula-I.

In another embodiment, the present invention provides simple process for the preparation of compounds of Formula-I, wherein the dehalogenation is performed in presence of aqueous carboxylic acid selected from the group consisting of p-aminobenzoic acid, p-hydroxybenzoic acid, benzoic acid, acetic acid, methyl benzoic acid, propionic acid and like thereof.

In another embodiment, the solvents are selected from lower alcohol, phenol, benzyl alcohol, ether, esters, amines, such as, ethanol, propanol, butanol, ethyl acetate, methyl acetate, polyethylene glycol (PEG), dimethyl ether, THF, ethyl amine and aniline.

In another embodiment, the warm temperature is maintained in the range of 40° C. to 130° C., particularly 50° C. to 80° C.

In another embodiment, the purification is caried out by filtration, centrifugation, evaporation, liquid-liquid extraction, crystallization, recrystallisation, titration, adsorption or any combination thereof.

In yet another preferred embodiment, the present invention provides novel therapeutically active moiety of Quaternary Ammonium Carboxylate Compounds represented by Formula Ia.

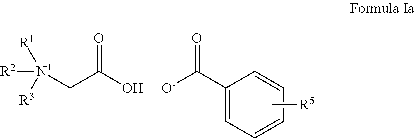

wherein R₁, R₂ and R₃ are similar or independently selected from the group consisting of H, (C1-C6)alkyl, hydroxy, C2-8 alkylenyl, alkyne, and C1-6 alkoxy, C2-4 alkylenyl, cycloalkyl, substituted or unsubstituted phenyl, aryl, arylalkyl, arylhalide.
wherein R₅ is selected from the group consisting of H, OH, methyl, ethyl methoxy, phenyl, benzyl, amine, (C3-C6) cycloalkyl.

In yet another preferred embodiment, the present invention provides novel therapeutically active quaternary ammonium carboxylate compounds of Formula-I which are represented as below:

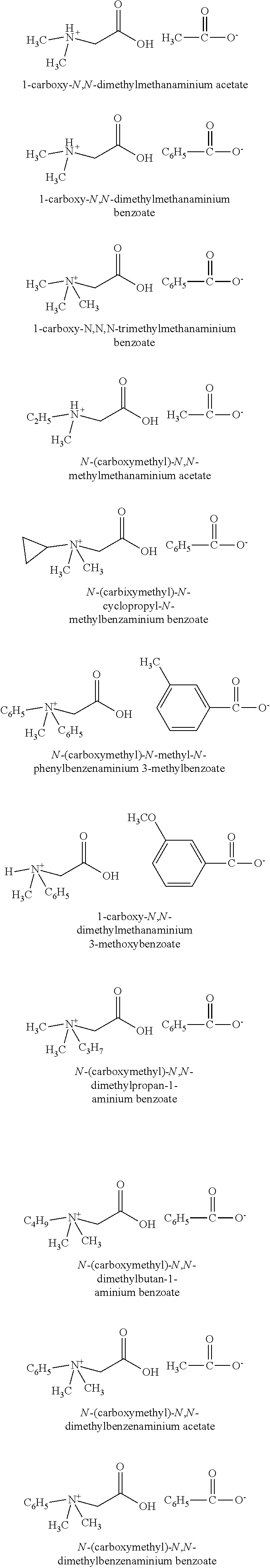

In yet another embodiment, the present invention provides a novel therapeutically active carboxylate of QACs represented by Formula-I with high potency and purity.

In yet another embodiment, the present invention provides novel molecular compounds comprising quaternary ammonium compounds bonded with carboxylate with strong ionic bond and therapeutic efficacy as compared to free base quaternary ammonium compounds and other known analogues of quaternary ammonium compounds.

In yet another embodiment, the present invention provides carboxylate of quaternary ammonium compounds (QACs) represented by Formula-I, useful in the treatment of certain neuropsychiatric disorders, neurological disorders and metabolic disorders or specific pathological conditions related to DSR dysfunctions.

The neuropsychiatric disorders include but are not limited to schizophrenia, schizophrenia, schizophreniform disorder, brief psychotic disorder, delusional disorder, schizotypal personality disorder, major depressive disorder, bipolar disorder, chronic hallucinatory psychosis, dissociative disorders, obsessive compulsive disorder, induced delusional disorder, posttraumatic stress disorder, menstrual psychosis, cycloid psychosis depression, mania (Bipolar disorder), visual hallucination, auditory hallucination, eating disorder, attention deficit hyperactivity disorder, Tourette's syndrome, other movement disorders, substance dependence (alcohol, cocaine), bipolar affective disorders, or unipolar affective disorder, adolescent conduct disorder.

The term ‘neurological disorders’ relates to any disorder of the nervous system (central and peripheral nervous system), structural, biochemical or electrical abnormalities in the brain, spinal cord or other nerves like cranial nerves, peripheral nerves, nerve roots, autonomic nervous system, neuromuscular junction, and muscles that can result in a range of symptoms. These disorders include but are not limited to epilepsy, Alzheimer's disease, dementia, cerebrovascular diseases including stroke, migraine and other headache disorders, multiple sclerosis, Parkinson's disease, neuroinfectious, brain tumours, traumatic disorders of the nervous system due to head trauma.

The term metabolic disorder refers to any disorder which occurs when abnormal chemical reactions in human body disrupt the metabolic processes. Metabolic syndrome is a cluster of conditions that occur together, increasing risk of heart disease, stroke, type 2 diabetes, high blood pressure, high blood sugar, abdominal obesity, liver health, NASH, ASH and abnormal cholesterol or triglyceride levels.

In another embodiment, the present invention provides novel therapeutically active substituted or unsubstituted Quaternary Ammonium Carboxylate Compounds (QACs) of Formula-I which are useful in the treatment of schizophrenia or psychosis where it acts as DAAO inhibitor.

Particularly, the DAAO inhibitor not only inhibits the degradation or oxidation of D-serine but also reduces neurotoxicity that leads to substantial decrease in oxidative stress, excitotoxicity and neuroinflammation.

In yet another embodiment, the present invention provides novel quaternary ammonium compounds of Formula I which act as NMDA modulator; as potent methyl donor; normalizes excess of active collapsin response mediator protein 2 (CRMP2) level; significantly inhibits glycine reuptake; significantly reduces carbonylated stress conditions; improves synaptic transmission in neuron thus improve neuro-development; influences nutrient transport and epigenetic regulation of fetal genes, which may subsequently normalize fetal growth and metabolism; and lowers the excessive plasma homocysteine level.

In another embodiment, the present invention provides compounds of Formula I normalizes cytoskeleton functions in neuronal development by regulating carbonyl stress in the body and the KIF₃ kinesin motor function in cells that synergistically leading to orderly neuronal cytoskeletal function.

In one embodiment, the compounds represented by Formula I, or pharmaceutically acceptable salts thereof, are formulated as a medicament, which preferably takes the form of therapeutically effective individual doses adjusted to the form of administration.

In one embodiment, the solid pharmaceutical preparations of compounds of Formula-I comprise fine particles or crystalline forms of compounds of Formula-I based on the method of purification.

Administration is simultaneous, sequential or separate, and provided in the form of a kit comprising the compounds of Formula-I, either combined together in a single medicament, or separately in the form of separate medicaments.

In one embodiment, the daily dose of a pharmaceutical composition disclosed herein varies over a wide range from about 0.1 mg to about 5000 mg; preferably, the dose is in the range of about 1 mg to about 1000 mg per day for an average human. For oral administration, the compositions are preferably provided in the form of tablets containing 0.1 to 1000 milligrams of formula I compounds for the symptomatic adjustment of the dosage to the subject to be treated.

Advantageously, compounds of Formula-I of the present disclosure are administered in a single daily dose or the total daily dosage is administered in divided doses of two, three or four times daily.

The term “therapeutically effective amount” denotes an amount that reduces the risk, potential, possibility or occurrence of a disease or disorder, or provides advanced alleviation, mitigation, and/or reduction or restoration or modulation, regulation of at least one indicator/biomarker (e.g., blood or serum CRP level), and/or minimize at least one clinical symptom related to neuropsychiatric disorder.

The term ‘subject in need thereof’ pertains to a subject preferably mammal, more preferably a human suffering or suspected with neuropsychiatric disorder.

In the context of the present invention, the term “treatment” refers to alleviate, mitigate, prophylaxis, attenuate, manage, regulate, modulate, control, minimize, lessen, decrease, down regulate, up regulate, moderate, inhibit, restore, suppress, limit, block, decrease, prevent, inhibit, stabilize, ameliorate or cure, heal neurological disorders observed in the patients.

Notably, the instant novel QACs carboxylate moiety is non-hazardous, non-toxic, and safe for human consumption without any severe adverse effects, therefore the present medicinal composition can also be used as preventive therapy/adjuvant therapy/add-on therapy/combination/adjunctive therapy in a subject in need thereof.

Certain compounds of the present invention exist in unsolvated forms as well as solvated forms, including hydrated forms. Further, some compounds of the present invention exist in multiple crystalline or amorphous forms (“polymorphs”). Compounds of the present invention are formulated in geometric or, enantiomeric or stereoisomeric forms.

In general, all physical forms disclosed herein are of use in the methods contemplated by the present invention and are intended to be within the scope of the present invention. Compound or pharmaceutically acceptable salts, hydrates, polymorphs or solvates of a compound intends the inclusive meaning of “or”, in those materials meeting more than one of the stated criteria are included, e.g., a material that is both a salt and a solvate is encompassed. Some of the crystalline forms of the compound exist as polymorphs and as such are intended to be included in the present disclosure. In addition, some of the compounds may form solvates with water (i.e., hydrates) or common organic solvents, and such solvates are intended to be encompassed by some embodiments.

In one embodiment, the present invention provides medicinal compositions comprising QACs of Formula I present in effective amount along with pharmaceutically acceptable excipients.

As used herein, the term “pharmaceutically acceptable carriers, diluents or excipients” is purported to mean, without limitation, any adjuvant, carrier, excipient, sweetening agent, diluents, preservative, dye/colorant, flavor enhancer, surfactant, wetting agent, dispersing agent, suspending agent, stabilizer, isotonic agent, solvent, emulsifier, or encapsulating agent, encapsulating polymeric delivery systems or polyethyleneglycol matrix, which is acceptable for use in the subject, preferably humans. Excipients also include, for example: antiadherents, antioxidants, binders, coatings, compression aids, disintegrants, dyes (colors), emollients, emulsifiers, fillers (diluents), film formers or coatings, fragrances, glidants (flow enhancers), lubricants, preservatives, sorbents, suspending or dispersing agents, sweeteners, surfactant, anticaking agent, food additives, or waters of hydration, salts.

In another embodiment, the present invention relates to medicinal composition comprising compounds of Formula-I prepared in a manner well known in the pharmaceutical art and administered by a variety of routes, depending upon whether local or systemic treatment is desired and upon the area to be treated. The preferable route of administration includes but is not limited to sublingual, rectal, topical, parenteral, nasal or oral.

In some embodiment, the present medicinal composition comprising compounds of Formula-I are administered to a subject in need thereof, in the form which is suitable for oral use, such as a tablet, capsule (in the form of delayed release, extended release, sustained release, enteric coated release); hard gelatin capsules, soft gelatin capsules in an oily vehicle, veg capsule, hard or soft cellulose capsule, granulate for sublingual use, effervescent or carbon tablets, aqueous or oily solution, suspension or emulsion, encapsulate, matrix, coat, beadlets, nanoparticles, caplet, granule, particulate, agglomerate, spansule, chewable tablet, lozenge, troche, solution, suspension, rapidly dissolving film, elixir, gel, tablets, pellets, granules, capsules, lozenges, aqueous or oily solutions, suspensions, emulsions, sprays or reconstituted dry powdered form with a liquid medium or syrup; for topical use including transmucosal and transdermal use, such as a cream, ointment, gel, aqueous or oil solution or suspension, salve, parch or plaster; for nasal use, such as a snuff nasal spray or nasal drops; for vaginal or rectal use, such as a suppository; for administration by inhalation, such as a finely divided powder or a liquid aerosol; for sub-lingual or buccal use, such as a tablet, capsule, film, spray. In another embodiment, the composition is formulated for parenteral use including intravenous, subcutaneous, intramuscular, intravascular, infusion, intraperitoneal, intracerebral, intracerebroventricular, or intradermal routes of administration.

In a further embodiment, the present composition is formulated in the form of age-appropriate pediatric oral dosage forms such as syrup, minitablets, chewable formulations, orodispersible films, orodispersible tablets.

Further, the present composition can be formulated in the form of age-appropriate pediatric oral dosage forms such as syrup, minitablets, chewable formulations, orodispersible films orodispersible tablets. It can also be prepared in the form of known food products.

Notably, the present composition is stable, non-hazardous, non-toxic and safe for human consumption without any side effects, therefore the present nutritional composition can also be used under preventive therapy/adjuvant therapy/add-on therapy/combination therapy in a subject in need thereof.

In another embodiment, the compounds of Formula I of the present invention are non-toxic, cost effective, enriched with nutrients or biomolecules; and provide safeguard against problems associated with neurotransmission without any adverse effect.

In some embodiments, the compounds of Formula-I can be formulated in medicaments by using pharmaceutically acceptable excipients such as diluents, binders, lubricants, solubilizing agents, surfactants, stabilizers, colors, flavoring agents, sweeteners, glidants, plasticizers, and other additives.

In another embodiment of the present invention, the diluents are selected from starches, hydrolyzed starches, partially pregelatinized starches, anhydrous lactose, cellulose powder, lactose monohydrate, sugar alcohols such as sorbitol, xylitol and mannitol, silicified microcrystalline cellulose, ammonium alginate, calcium carbonate, calcium lactate, dibasic calcium phosphate (anhydrous/dibasic dehydrate/tribasic), calcium silicate, calcium sulphate, cellulose acetate, corn starch, pregelatinized starch, dextrin, β-cyclodextrin, dextrates, dextrose, erythritol, ethyl cellulose, fructose, fumaric acid, glyceryl palmitostearate, magnesium carbonate, magnesium oxide, maltodextrin, maltose, medium-chain triglycerides, polydextrose, polymethacrylates, sodium alginate, sodium chloride, sterilizable maize, sucrose, sugar spheres, talc, trehalose, xylitol, vehicles like petrolatum, dimethyl sulfoxide and mineral oil or the like.

In some embodiment of the invention, the diluent in the composition/formulation is present in a range of 1% to 30% by weight of the total composition/formulation.

In yet another embodiment of the invention, the binder is selected from disaccharides such as sucrose, lactose, polysaccharides and their derivatives like starches, cellulose, or modified cellulose such as microcrystalline cellulose and cellulose ethers such as hydroxypropyl cellulose (HPC); hydroxypropyl methyl cellulose (HPMC); sugar alcohols such as xylitol, sorbitol, or mannitol; protein like gelatin; synthetic polymers such as polyvinylpyrrolidone (PVP), polyethylene glycol (PEG), starch, acacia, agar, alginic acid, calcium carbonate, calcium 15 lactate, carbomers, carboxymethylcellulose sodium, carrageenan, cellulose acetate phthalate, chitosan, co-povidone, corn starch, pregelatinized starch, cottonseed oil, dextrates, dextrin, dextrose, ethyl cellulose, guar gum, hydrogenated vegetable oil, mineral oil, hydroxyethyl cellulose, hydroxymethyl cellulose, hydroxyl ethyl methyl cellulose, hydroxypropyl cellulose, inulin, cellulose, methyl cellulose, polyvinylpyrrolidone and polyethylene glycol, lactose, liquid 20 glucose, hypromellose, magnesium aluminum silicate, maltodextrin, maltose, methyl-cellulose, microcrystalline cellulose, pectin, poloxamer, polydextrose, polymethacrylates, povidone, sodium alginate, stearic acid, sucrose, sunflower oil, various animal vegetable oils, and white soft paraffin, paraffin, flavorants, colorants and wax.

In further embodiment of the present invention, the binder in the composition/formulation is present in a range of 0.1 to 40% by weight of the composition/formulation.

In another embodiment of the present invention, the lubricant is selected from magnesium stearate, zinc stearate, calcium stearate, glycerin monostearate, glyceryl behenate, glyceryl palmitostearate, hydrogenated castor oil, hydrogenated vegetable oil, light mineral oil, magnesium lauryl sulphate, medium-chain triglycerides, mineral oil, myristic acid, palmitic acid, poloxamer, polyethylene glycol, sodium benzoate, sodium chloride, sodium lauryl sulphate, sodium stearyl fumarate, stearic acid, talc, potassium, or sodium benzoate or the like.

In some embodiment of the present invention, the lubricant in the composition/formulation is present in a range of 0.1% to 10.0% by weight of the total composition/formulation.

In another embodiment of the present invention, the solubilizing agent is selected from polysorbate 80, sodium lauryl sulphate, anionic emulsifying wax, nonionic emulsifying wax, glyceryl monooleate, phospholipids, polyoxyethylene alkyl ethers, polyoxyethylene castor oil derivatives, polyoxyethylene sorbitan fatty acid esters, polyoxyethylene stearates, polyoxylglycerides, sorbitan esters, triethyl citrate, vitamin E, polyethylene glycol succinate, microcrystalline cellulose, carboxymethylcellulose sodium, diethanolamine, ethylene glycol palmitostearate, glycerin monostearate, hypromellose, hypromellose, acetate succinate, lecithin, polyethylene alkyl ethers, aluminum oxide, poly(methylvinyl ether/maleic anhydride), calcium carbonate, crospovidone, cyclodextrins, fructose, hydroxpropyl betadex, oleyl alcohol, povidone, benzalkonium chloride, benzethonium chloride, benzyl alcohol, benzyl benzoate, cetylpyridinium chloride, inulin, meglumine, poloxamer, pyrrolidone, sodium bicarbonate, starch, stearic acid, sulfobutylether beta cyclodextrin, tricaprylin, triolein, docusate sodium, glycine, alcohol, self-emulsifying glyceryl monooleate, cationic benzethonium chloride, cetrimide, xanthan gum, lauric acid, myristyl alcohol, butylparaben, ethylparaben, 15 methylparaben, propylparaben, sorbic acid or the like.

In another embodiment of the present invention, the amount of solubilizing agent or surfactant in the composition/formulation ranges from 0.1% to 10% by weight of the composition/formulation.

In a preferred embodiment of the present invention, the solubilizing agent or surfactant is present in a range of 0.1% to 5.0% by weight of the composition/formulation.

In some embodiment of the present invention, the glidant is selected from colloidal silicon dioxide, magnesium stearate, fumed silica (colloidal silicon dioxide), starch, talc, calcium phosphate tribasic, cellulose powdered, hydrophobic colloidal silica, magnesium oxide, zinc stearate, magnesium silicate, magnesium trisilicate, silicon dioxide or the like.

In another embodiment of the present invention, the glidant in the composition/formulation is present in a range of 0.1% to 5.0% by weight of the total composition/formulation.

In some embodiment of the present invention, the stabilizers are selected from the group consisting of alginate, agar, carrageen, gelatin, guar gum, gum arabic, locust bean gum, pectin, starch, xanthan gum, trehalose and likewise.

In some embodiment of the present invention, the stabilizer in the composition/formulation is present in a range of 0.1% to 8.0% by weight of the total composition/formulation. In some embodiment of the invention, the plasticizers are added to coating formulations selected from the group propylene glycol, glycerol, glyceryl triacetate (triacetin), triethyl citrate, acetyl triethyl citrate, diethyl phthalate, actetylated monoglycerides, castor oil, mineral oil and like thereof.

In some embodiment of the present invention, the plasticizer in the composition/formulation is present in a range of 0.1% to 5.0% by weight of the total composition/formulation.

In some embodiment of the present invention, the solvent is selected from water, alcohol, isopropyl alcohol, propylene glycol, mineral oil, benzyl alcohol, benzyl benzoate, flavored glycol, carbon dioxide, castor oil, corn oil (maize), cottonseed oil, dimethyl ether, albumin, dimethylacetamide, ethyl acetate, ethyl lactate, medium-chain triglycerides, methyl lactate, olive oil, peanut oil, polyethylene glycol, polyoxyl, castor oil, propylene carbonate, pyrrolidone, safflower oil, sesame oil, soybean oil, sunflower oil, water-miscible solvents, organic polar or non-polar solvents or mixtures thereof.

In a preferred embodiment of the present invention, the solvent in the composition/formulation is used in a quantity sufficient to make the weight of the composition/formulation 100% by weight.

The additional additives include a polymer, a plasticizer, a sweetener, and a powdered flavor, a preservative, a colorant, a surfactant, and other excipients. The powdered flavor composition includes a flavourant associated with a solid carrier. Coating materials such as synthetic polymers, shellac, corn protein (zein) or other polysaccharides, gelatin, fatty acids, waxes, shellac, plastics, and plant fibers and like thereof are used.

In a preferred embodiment of the present invention, the additives are used in a range of 1 to 20% w/w of unit dose.

In yet another embodiment, the present invention provides a medicinal composition comprising compounds of Formula I along with pharmaceutical excipients, wherein the pharmaceutical excipients are selected from a diluent, a binder, a lubricant, a glidant, an additive, a surfactant, a stabilizer or mixtures thereof.

In a preferred embodiment, the diluent is present in a range of 1 to 30%; the binder present is present in a range of 0.1 to 25%; the lubricant is present in a range of 0.1 to 10.0%; the glidant is present in a range of 0.1 to 5.0%; the additive is present in a range of 1 to 10%; the surfactant is present in a range of 0.1 to 5.0%; the stabilizer is present in a range of 0.1 to 5.0%; and the plasticizer is present in a range of 0.1 to 5.0%; by weight of total composition. In a preferred embodiment, the present medicinal composition/formulation is formulated for oral administration. Specifically, the solid medicinal compositions, are in the form of tablets, capsules, pills, hard capsules filled with liquids or solids, soft capsules, sachets, powders, granules, suspensions, solutions, or modified release formulations. Formulations of the present 10 invention suitable for oral administration are presented as discrete units such as capsules (e.g., soft-gel capsules, hard-gel capsule), cachets or tablets each containing a predetermined amount of the active ingredient; as a powder or granules; as a solution or a suspension in an aqueous liquid or a non-aqueous liquid, syrup; or as an oil-in-water liquid emulsion or a water-in-oil liquid emulsion.

The magnitude of a prophylactic or therapeutic dose typically varies with the nature and severity of the condition to be treated and the route of administration. The dose, and perhaps the dose frequency, will also vary according to the age, body weight and response of the individual patient. In general, the total daily dose (in single or divided doses) ranges from about 1 mg per day to about 2500 mg per day, preferably about 10 mg per day to about 1000 mg per day. In some embodiments, the total daily dose can range from about 5 mg to about 4000 mg per day, and preferably about 5 mg to about 2000 mg per day.

In another embodiment, the present invention provides a method for treating neuropsychiatric disorders in a subject in need thereof. The method comprises administering an oral dose of a therapeutically effective amount of a medicinal composition comprising compounds of Formula-I along with pharmaceutically acceptable excipients.

In certain embodiments, the invention provides the potent synergistic medicinal composition wherein the effective unit dose for an oral administration is formulated in a range of 1 to 1000 mg.

It is further recommended that children, patients over 60 years old, initially receive low doses and that the dosage be titrated based on individual physiological responses and/or pharmacokinetics. It can be necessary to use dosages outside these ranges in some cases, as will be apparent to those in the art. The present composition can be used as infant formula as well as adult formula by varying the concentration of active ingredients. Further, it is noted that the dietician or nutritionist or certified physician knows how and when to interrupt, adjust or terminate therapy in conjunction with an individual patient's response.

The use of any and all examples, or exemplary language (e.g., such as) provided herein, is intended merely to better illuminate the invention and does not pose a limitation on the scope of the invention unless otherwise claimed.

While in the foregoing specification this invention has been described in relation to certain embodiments thereof, and many details have been put forth for the purpose of illustration, it will be apparent to those skilled in the art that the invention is susceptible to additional embodiments and that certain of the details described herein can be varied considerably without departing from the basic principles of the invention.

The invention may be further illustrated by the following examples, which are for illustrative purposes only and should not be construed as limiting the scope of the invention in anyway. The present invention is not to be limited in terms of the particular embodiments described in this application, which are intended as single illustrations of individual aspects of the invention. Functionally equivalent compositions and treatments within the scope of the invention, in addition to those enumerated herein, will be apparent to those skilled in the art from the foregoing description and examples. Such modifications and variations are intended to fall within the scope of the appended claims. The contents of each reference, patent and patent application cited in this application is hereby incorporated by reference in its entirety.

The magnitude of a prophylactic or therapeutic dose typically varies with the nature and severity of the condition to be treated and the route of administration. The dose, and perhaps the dose frequency, will also vary according to the age, body weight and response of the individual patient.

In general, the total daily dose (in single or divided doses) ranges from about 1 mg per day to about 5000 mg per day, preferably about 10 mg per day to about 1500 mg per day.

In some embodiment, the total daily dose can be administered in the range of about 1 mg to about 3000 mg per day, and preferably about 5 mg to about 1000 mg per day. Having described the basic aspects of the present invention, the following non-limiting examples illustrate specific embodiments thereof. Those skilled in the art will appreciate that many modifications may be made in the invention without changing the essence of invention.

The invention may be further illustrated by the following examples, which are for illustrative purposes only and should not be construed as limiting the scope of the invention in anyway.

The present disclosure is therefore to be considered as in all respects illustrative and not restrictive, the scope of the invention being indicated by the appended claims and examples, and all changes or alterations which come within the ambit of equivalency are intended to be encompassed therein.

### EXAMPLES

### Example 1

### Synthesis of 1-carboxy-N—N—N-trimethylmethanaminium benzoate

To 100 ml round bottom flask with a magnetic stirrer, condenser and thermometer was housed, added 10 ml water, with stirring, white crystalline benzoic acid (10 g, 81 mmol) was added to (15.4 g, 0.1 mole) of 1-carboxy-N—N—N-trimethylmethanaminium chloride, made it molten entirely, dripped isopropanol (15 mL). The reaction mixture was heated at reflux for 4 hours. When the reaction was completed, the mixture was allowed to cool. The mixture was filtered and isopropanol was removed in vacuo to afford the product as a viscous, semi-solid (23 g).

The crude product was further purified under 60° C.-90° C. conditions, water was made into the saturated solution of 1-carboxy-N—N—N-trimethylmethanaminium benzoate, heated to and boil, be chilled to 0-25° C. then and carry out recrystallization. After crystallization the solid dry form of 1-carboxy-N—N—N-trimethylmethanaminium benzoate obtained with purity 95-99% and yield 80-90%.

a) The NMR characterization of 1-carboxy-N—N—N-trimethylmethanaminium benzoate was performed in DMSO: Number of protons of benzoate was confirmed, whereas number of protons of 1-carboxy-N—N—N-trimethylmethanaminium was observed on higher. (—CH₃ proton observed 12.13 instead of 9 protons & —CH₂ proton observed 2.95 instead of 2).

H₁ NMR (400 MHz, CD₃OD)

δ ppm: 3.265 (s, 12H, 3× methyl, 1× methyl of MeOD), 3.827 (s, 2H, Methylene), 7.437-7.475 (t, 2H, meta Ar—H), 7.556-7.600 (t, 1H, para Ar—H), 7.556-7.600 (d, 2H, ortho Ar—H) b) The NMR characterization of 1 carboxy N—N—N trimethylmethanaminium benzoate was performed in Methanol: Number of protons of benzoate was confirmed, whereas number of protons of 1 carboxy N—N—N trimethylmethanaminium was observed on higher. (—CH₃ proton observed 14.82 instead of 9 protons & —CH₂ proton observed 3.23 instead of 2).

### Example 2: Benzoic Acid Content by HPLC

Instrument: HPLC system equipped with UV detector, Sampler cooler and Auto sampler with operating software.

For HPLC analysis AR or HPLC grade solvents like ammonium acetate, glacial acetic acid, acetonitrile was used.

### Chromatographic Conditions:

Column: GL Science Inertsil C8 150×4.6 mm, 5.0 μm or equivalent
Flow rate: 1.0 mL/minute
Detection: 230 nm
Injection: 20 μL
Column Temperature: 30° C.
Sampler cooler Temperature: 25° C.
Run time: 25 minutes
Buffer solution: 50 mM ammonium acetate was prepared in water, mixed well. The pH was adjusted to pH 4.00±0.05 by glacial acetic acid. Filtered through 0.45μ nylon filter and degassed it.
Mobile phase A: Buffer solution.
Mobile phase B: Acetonitrile.
Gradient Programme:

| Time (Mins) | Mobile phase A (%, v/v) | Mobile phase B (%, v/v) |
|---|---|---|
| 0.01 | 90 | 10 |
| 2 | 90 | 10 |
| 15 | 40 | 60 |
| 20 | 40 | 60 |
| 21 | 90 | 10 |
| 25 | 90 | 10 |

Diluent: Mobile Phase A: Mobile Phase B 90:10 v/v. Mix well and degas it.

### Preparation of Solutions:

Blank solution: Diluent.
Standard solution-A: “Benzoic acid” Standard of 0.1 mg/mL

For Example: Accurately weighted about 50 mg of benzoic acid Standard into 50 mL volumetric flask, sonicated to dissolve and dilute up to the volume with diluent. 5 mL of this solution was transferred in 50 mL volumetric flask and diluted up to the volume with diluent.

Test solution: 1 carboxy N—N—N trimethylmethanaminium benzoate sample of 0.1 mg/mL

For Example: Accurately weighted about 50 mg of sample into 50 mL volumetric flask, sonicated to dissolve and diluted up to the volume with diluent. 5 mL of this solution was transferred in 50 mL volumetric flask and diluted up to the volume with diluent.

Procedure: Separately injected equal volume of solutions as per sequence of injection as given below into the chromatograph and recorded the peak responses and checked the system suitability requirements. The peak was interpreted only due to Benzoic acid.

The Retention time of Benzoic acid peak should be 10.4 (+10%) mins.

Benzoic acid content by HPLC-45.47%

### Example 3:1-Carboxy-N—N—N-trimethylmethanaminium Content by Potentiometry

Standardization of 0.1N Perchloric acid solution: 0.350 g of Potassium hydrogen phthalate was dissolved in 50.0 mL of Glacial acetic acid warm gently if necessary. Allowed to cool, titrated the solution against the 0.1N Perchloric acid solution and determined the end point by Potentiometry.

1 mL of 0.1 N Perchloric acid was equivalent to 20.42 mg of C₈H₅KO₄.

Normality of 0.1 N Perchloric acid solution:

N=(Weight of Potassium hydrogen phthalate in g×0.1)/(Volume of Perchloric acid consumed in mL×0.020422)

Procedure for Assay: Accurately weighted 0.20 g of sample and transferred into a 150.0 mL of beaker. 50.0 mL of Glacial acetic acid was added and sonicated to dissolve. The solution was titrated against 0.1N Perchloric acid and determined the end point by Potentiometry. A blank titration was performed simultaneously.

Each mL of 0.1N Perchloric acid was equivalent to 11.711 mg of C₅H₁₁NO₂.

1 carboxy N—N—N trimethylmethanaminium content by potentiometry-56.48%

### Example 4: Dissociation of 1 carboxy N—N—N trimethylmethanaminium benzoate

Sample Preparation: 750 mg sample was dissolved in 250 mL of respective buffer solution and kept in orbital shaker at 37° C. for 30 minutes, 1 hour and 2 hours in separate flask.

### A. Concentration in %

| | | 1 carboxy N-N-N | | | |
|---|---|---|---|---|---|
| | | trimethylmethanaminium, % | | Benzoic acid, % | |
| | | pH 1.2 | pH 6.8 | pH 1.2 | pH 6.8 |
| | | (0.1N | (Phosphate | (0.1N | (Phosphate |
| Time Point | | HCl) | Buffer) | HCl) | Buffer) |
| Control | | 70 | | 36 | |
| Initial | | 67 | 69 | 34 | 36 |
| 30 | Minutes | 68 | 71 | 41 | 40 |
| 1 | Hours | 69 | 70 | 41 | 40 |
| 2 | Hours | 70 | 71 | 41 | 40 |

### B. %, Dissociation:

| | | %, Dissociation of 1 carboxy | | %, Dissociation | |
|---|---|---|---|---|---|
| | | N-N-N trimethylmethanaminium | | of Benzoic acid | |
| | | pH 1.2 | pH 6.8 | pH 1.2 | pH 6.8 |
| | | (0.1N | (Phosphate | (0.1N | (Phosphate |
| Time Point | | HCl) | Buffer) | HCl) | Buffer) |
| Initial | | 97 | 96 | 94 | 97 |
| 30 | Minutes | 98 | 98 | 115 | 109 |
| 1 | Hours | 100 | 97 | 115 | 109 |
| 2 | Hours | 100 | 99 | 114 | 108 |

Dissociation study of 1-carboxy-N—N—N-trimethylmethanaminium benzoate was performed in 0.1N HCl for 2 hours at room temperature.

1-carboxy-N—N—N-trimethylmethanaminium benzoate was completely dissociated in 1.2 pH and 6.8 pH.

1. A quaternary ammonium carboxylate compound of Formula I: 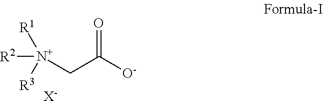
wherein R₁, R₂ and R₃ are same or independently selected from the group consisting of H, OH, (C1-C9)alkyl, alkylene, alkyne, (C1-C9)-O—, heterocyclic group, —COOR, halide,
substituted or unsubstituted cycloalkyl, aryl and arylalkyl;
wherein X is 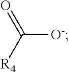
wherein R₄ is selected from the group consisting of H, OH, (C1-C9)alkyl, alkylene, alkyne, aryl, aralkyl, (C3-C8) cycloalkyl, (C1-C9)-O—, heterocyclic group, —COOR, halide and 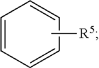
wherein R is lower alkyl;
wherein R₅ is selected from the group consisting of H, OH, alkyl, alkoxy, aryl, biphenyl, heterocyclic and cycloalkyl compounds.

2. The quaternary ammonium carboxylate compound as claimed in claim 1, wherein the compound is of Formula 1a: 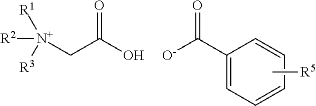
wherein R₁, R₂ and R₃ are same or independently selected from the group consisting of H, (C1-C6)alkyl, hydroxy, C2-8 alkylenyl, and C1-6 alkoxy, C2-4 alkylenyl, cycloalkyl, substituted or unsubstituted phenyl, aryl, arylalkyl and arylhalide;
wherein R₅ is selected from the group consisting of H, OH, methyl, ethyl methoxy, phenyl, benzyl, amine and (C3-C6) cycloalkyl.

3. The quaternary ammonium carboxylate compound as claimed in claim 1, wherein the compound is:

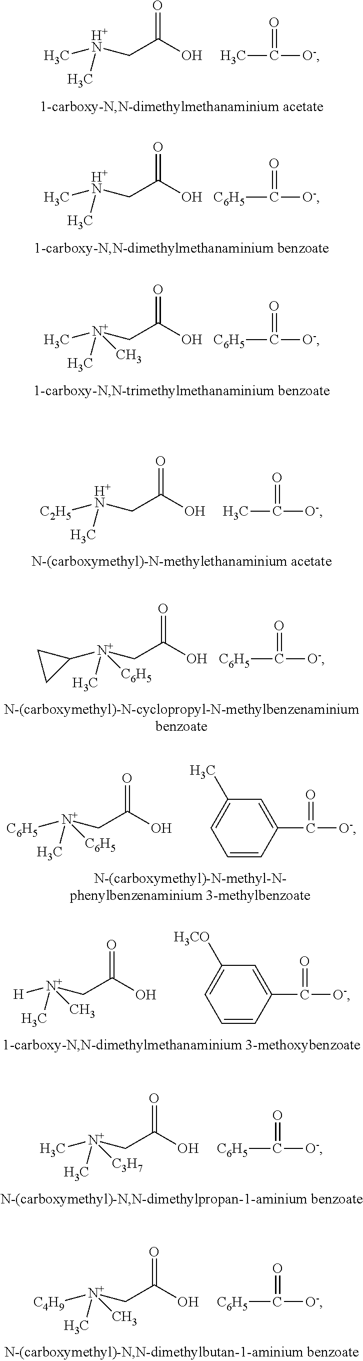

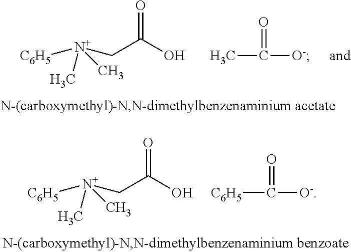

4. The quaternary ammonium carboxylate compound as claimed in claim 1, wherein the compound is useful in the treatment of neuropsychiatric disorders selected from the group consisting of schizophrenia, schizophrenia, schizophreniform disorder, brief psychotic disorder, delusional disorder, schizotypal personality disorder, major depressive disorder, bipolar disorder, chronic hallucinatory psychosis, dissociative disorders, obsessive compulsive disorder, induced delusional disorder, posttraumatic stress disorder, menstrual psychosis, cycloid psychosis depression, mania or bipolar disorder, visual hallucination, auditory hallucination, eating disorder, attention deficit hyperactivity disorder, Tourette's syndrome, other movement disorders, dependence on alcohol and cocaine, bipolar affective disorders or unipolar affective disorder and adolescent conduct disorder.

5. A process for preparation of a compound of Formula-I, comprising:
i) dehalogenation quaternary ammonium compound of Formula-I in presence of an organic solvent at warm condition to obtain a crude quaternary ammonium carboxylate compound;
ii) purification of the crude quaternary ammonium carboxylate compound at a temperature in the range of 10° C.-130° C. to obtain high yield and purity of the compound of Formula-I.

6. The process as claimed in claim 5, wherein the dehalogenation is performed in presence of aqueous carboxylic acid selected from the group consisting of p-aminobenzoic acid, p-hydroxybenzoic acid, benzoic acid, acetic acid, methyl benzoic acid and propionic acid.

7. The process as claimed in claim 5, wherein the organic solvent is selected from the group consisting of ethanol, propanol, butanol, ethyl acetate, methyl acetate, polyethylene glycol (PEG), dimethyl ether, THF, ethyl amine and aniline.

8. The process as claimed in claim 5, wherein the warm condition is maintained at a temperature in the range of 40° C.-130° C.

9. A composition comprising a compound of Formula-I and at least one pharmaceutically acceptable excipient.

10. The composition as claimed in claim 9, wherein the pharmaceutically acceptable excipient is selected from the group consisting of diluent, binder, lubricant, glidant, additive, surfactant, stabilizer and plasticizer; wherein the diluent is present in a range of 1 to 30%;
wherein the binder present is present in a range of 0.1 to 25%; wherein the lubricant is present in a range of 0.1 to 10.0%; wherein the glidant is present in a range of 0.1 to 5.0%;
wherein the additive is present in a range of 1 to 10%; wherein the surfactant is present in a range of 0.1 to 5.0%; wherein the stabilizer is present in a range of 0.1 to 5.0%; and
wherein the plasticizer is present in a range of 0.1 to 5.0% by weight of the composition.
